(19) United States
(12) Patent Application Publication
(10) Pub. No.: US 2023/0032649 A1
(43) Pub. Date: Feb. 2, 2023

(54) CRANIOFACIAL IMPLANTS FOR NEUROPLASTIC SURGERY

(71) Applicant: Acumed LLC, Hillsboro, OR (US)

(72) Inventor: Chad GORDON, Cockeysville, MD (US)

(21) Appl. No.: 17/801,652

(22) PCT Filed: Feb. 24, 2021
(86) PCT No.: PCT/US2021/019340

§ 371 Date: Aug. 23, 2022

Related U.S. Application Data
(60) Provisional application No. 62/980,558, filed on Feb. 24, 2020.

(51) Int. Cl. A61F 2/28

(52) U.S. Cl. CPC A61F 2/2875; A61B 2034/105

(57) ABSTRACT

Craniofacial implants for neuroplastic surgery structured for filling cranial bone voids in the skull designed, shaped and manufactured to address problems including cranioplasty failure, soft tissue thinning above the implant, overlying scalp atrophy leading to contour irregularities and/or scalp breakdown with exposure/infection, ineffective pre-fabricated shapes with sharp corners requiring manually bending or cutting the implants for proper form/shape, and additional operative time and anesthesia morbidity, and a need to spend time hand-trimming prong edges along the perimeter to ensure an absence of sharp edges and/or corners capable of injuring the scalp/soft tissue above, leading to chronic pain. The craniofacial implants for neuroplastic surgery are configured to have an improved shape absent corners, a smoother contour, a shape addressing co-existing soft tissue temporal deformity/atrophy, and/or a smoother, frictionless coating in order to prevent complications leading to suboptimal outcomes and implant removal by way of prior art implants.

### BACKGROUND

In 1965, titanium mesh implants were first described in a small case series by Simpson [Titanium in Cranioplasty. J Neurosurgery 1965]. Since then, evidence has shown titanium to be a safe, strong, and reliable material with high amounts of biocompatibility. Currently, titanium mesh implants are a popular choice amongst neurosurgeons and reconstructive surgeons alike for several attractive features such as “off-the-shelf” availability, minimal radiaographic artifact, malleability, strength, and light weight. However, such implants have been reported by many groups to cause thinning, erosion, and premature atrophy of the overlying scalp/soft tissue due to its inherent design flaws from the 1960's, thereby increasing one's risk for exposure, extrusion, secondary infection, and ultimate removal (i.e. failure). For example, in article entitled “Comparison of Polyetheretherketone and Titanium Cranioplasty after Decompressive Craniectomy” by Thien et al (World Neurosurgery, 2015), the authors found that 27 of 108 titanium implants led to failure due to the aforementioned reasons, such as having a harsh, course contour and mesh-“imprinted design with sharp circular edges” leading to repetitive microinjury (25%). Similarly, Maqbool et al reported significant scalp/soft tissue atrophy in 44% of their patients with titanium mesh cranioplasties and 14% extrusion rate of the implant leading to failure (i.e. 1 out of 7 patients lost their craniofacial implant due to extrusion). [Maqbool, et al. Risk Factors for Titanium Mesh Implant Exposure Following Cranioplasty. J Craniofacial Surgery 2018.].

Exposure is a particularly serious and adverse complication that not only poses a risk of secondary infection and ultimate removal, leaving one with a persistent skull defect, but it can also lead to life-threatening sequelae like meningitis, epidural epyema, and/or death if not treated in a timely fashion. Therefore, any and all efforts to prevent injury and abrasion to the overlying soft tissue should take high priority moving forward. Unfortunately, the exact reasons for titanium mesh implant exposure have not been thoroughly investigated by the field and thus contributes to a remaining flaw in the current state of the art and implant design [Maqbool, et al. Risk Factors for Titanium Mesh Implant Exposure Following Cranioplasty. J Craniofacial Surgery 2018.]. In this article, the authors found a greater odds of titanium mesh exposure when soft tissue/scalp thinning became present external to the implant (defined as greater than 50% thickness versus the contralateral, non-operated side] and more importantly, that soft tissue thinning was found to be a “temporal phenomenon” and ultimately related to its “chronic friction with the sharp titanium edges” to the lateral face and scalp/soft tissue above. Thus, a proven association to the temporal area anatomy is not only “eye-opening” to the field, it is also critically important given the prevalence of temporal deformity post-neurosurgery, along with its accompanying social stigma, and why new designs an shaped are much needed. As such, the field of neuro-cranial reconstruction needs not only a safer improvement to the current titanium mesh implant in terms of its coarseness and “sand-paper like” edges by way of a novel coating/design, it also needs an improved pre-bent curvature to better address the temporal region and thereby provide craniofacial symmetry. Furthermore, in this same article, the group also describes a suboptimal need to “manually shape” the titanium mesh implants during surgery, which results in numerous drawbacks. They also conclude that “conversely, the exposure of smooth-contour implants is rare” and “that erosion of overlying soft tissues and implant exposure is definitely observed with titanium mesh and is likely causally related to the mesh pattern of the implants.”

In another article by Kwiecien et al from 2018, entitled “Long-term Outcomes of Cranioplasty” [Annals of Plastic Surgery], the group reported that they unexpectedly found—during retrospective review of 401 patients over 12 years—“a number of titanium mesh extrusions and hardware failures.” Interestingly, the team further reported that this dreadful complication (i.e. titanium mesh extrusion) was, in fact, the most common complication found altogether during their entire study timeline—even when all the other potential complications such as bleeding and infection related to brain surgery and skull reconstruction are considered. In conclusion, they noted “that titanium mesh exposure occurred at a surprisingly high complication rate of 42.2%”. As such, it is critical for the neurosurgical community to advance this field to drastically improve the state of the art.

Further, a paper entitled “Long-term Effect of Cranioplasty on Overlying Scalp Atrophy” by Kwiecien et al [Plast Reconstr Surg 2020], is quite relevant to this important subject. The authors of this paper are emphatic in reporting that the temporal hollowing deformity is a well-known, common complication in this type of surgery. Furthermore, the authors state that its reported incidence after decompressive craniectomy (i.e. the original removal of bone to allow for temporary brain swelling and necessitating delayed reconstruction with craniofacial implant), is as high as 100%—again, attesting to the need for improvement in the field.

Additionally, in their 10-year database review spanning 2003 to 2013 at the Cleveland Clinic, the authors found that “scalp/soft tissue atrophy overlying a titanium mesh implant continued to progress over time, unlike solid alloplastic implants and/or autologous bone with high statistical significance (P<0.01)”. Per this article, univariate logistic regression analysis in 101 consecutive skull reconstruction patients again found “titanium mesh material as an independent risk factor leading to scalp thinning”—well align with the previously noted peer-reviewed, journal articles. Additionally, authors also state quite emphatically that “the strongest independent predictor of scalp (i.e. soft tissue) atrophy was the use of titanium mesh material (P<0.01)”, again with strong statistical significance. Hence, it is clear that the current state of the art design of titanium mesh, with sharp edges and a course contour, is flawed and in need of improvement.

Thus, existing craniofacial implants suffer from a variety of drawbacks, including difficulty in persistent soft tissue temporal hollowing, soft tissue thinning, overlying scalp atrophy, a labor intense necessity to form the implants, additional operative expense related to modification at time of placement, challenges during the implantation process, and increased chances of exposure thereby leading to inevitable infection and removal.

Further, existing craniofacial implants, including 3D-printed materials like titanium mesh, poly-methylmethcrylate (PMMA), PEEK, and/or porous polyethylene are typically expensive and do not perform well in patients with suboptimal scalp healthiness/thickness like instances of chronic tobacco exposure, previous irradiation and/or extensive scarring, as well as areas most prone to infection, such as in skull defect sites after previously-failed craniofacial implants and polymicrobial contamination. In such areas, infection has been known to occur 35%+ of the time following repeat craniofacial implant surgery.

### SUMMARY

A craniofacial implant, method of making the implant, method of using and implanting the implant, and related manufacturing and surgical methods and systems. The method for making the craniofacial implant can include determining a size of a bony defect and/or void in a craniofacial region, selecting an implant formed of a piece of pre-manufactured titanium mesh of a size that covers the bony defect and/or void and at least a portion of the bone surrounding the bony defect and/or void, wherein the implant is pre-sized to common craniectomy dimensions and pre-contoured using a normal radius of curvature for a human skull; and sterilizing the implant.

In another embodiment, a craniofacial implant for neuroplastic surgery may be provided. The craniofacial implant can include a piece of titanium mesh, the titanium mesh formed so as to be substantially round or otherwise without corners, the titanium mesh molded and shaped to substantially match the radius of curvature of a human skull, the titanium mesh further comprising a smooth surface and a plurality of screw holes formed with countersinking.

### BRIEF DESCRIPTION OF THE DRAWINGS

The accompanying drawings illustrate various embodiments of systems, methods, and embodiments of various other aspects of the disclosure. Any person with ordinary skills in the art will appreciate that the illustrated element boundaries (e.g. boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another, and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1 is an exemplary flowchart showing a method of forming a craniofacial implant.

FIG. 2 is an exemplary diagram showing an embodiment of a craniofacial implant in a first size.

FIG. 3 is an exemplary diagram showing an embodiment of a craniofacial implant in a second size.

FIG. 4 is an exemplary diagram showing an embodiment of a craniofacial implant in a third size.

FIG. 5 is an exemplary diagram showing embodiments of craniofacial implants.

FIG. 6A is an exemplary diagram showing an implant next to a bony void of a skull.

FIG. 6B is an exemplary diagram showing an implant covering a bony void of a skull.

### DETAILED DESCRIPTION OF THE EMBODIMENTS

Aspects of the invention are disclosed in the following description and related drawings directed to specific embodiments of the invention. Alternate embodiments may be devised without departing from the spirit or the scope of the invention. Additionally, well-known elements of exemplary embodiments of the invention will not be described in detail or will be omitted so as not to obscure the relevant details of the invention. Further, to facilitate an understanding of the description discussion of several terms used herein follows.

As used herein, the word “exemplary” means “serving as an example, instance or illustration.” The embodiments described herein are not limiting, but rather are exemplary only. It should be understood that the described embodiments are not necessarily to be construed as preferred or advantageous over other embodiments. Moreover, the terms “embodiments of the invention”, “embodiments” or “invention” do not require that all embodiments of the invention include the discussed feature, advantage or mode of operation.

Exemplary embodiments described herein may relate to craniofacial implants and titanium mesh used for craniofacial implants. Such titanium mesh implants may be desirable in locations, for example, where chronic infections are known to occur.

A titanium mesh implant can be formed and utilized in any of a variety of manners. Current methods include “off-the-shelf” or 3D-printed titanium mesh products for which are universal in design. They are typically flat, typically square or rectangular, typically have unnecessary corners needing trimming, and most importantly, have a rough, “sand-paper” like surface causing high friction and micro-injury to the supple scalp and soft tissue above, as noted previously.

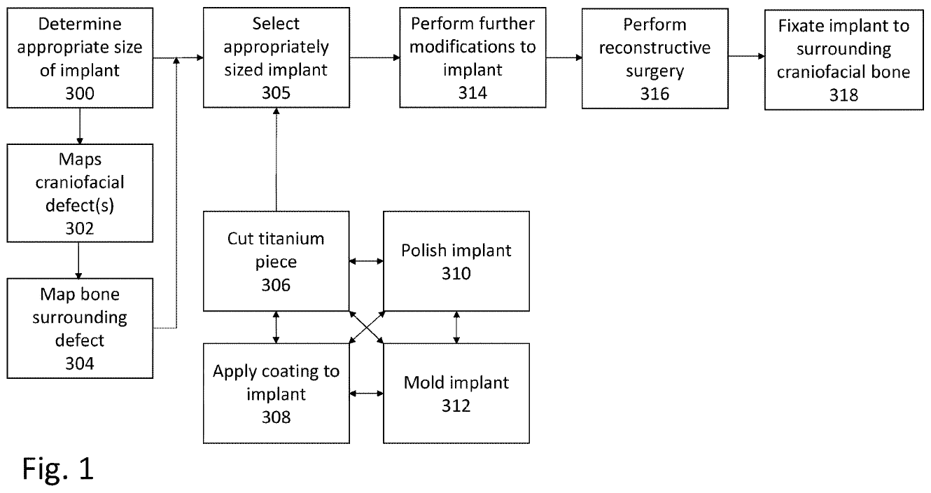

Thus, as shown in exemplary FIG. 1, a method for providing a cranial implant for neuroplastic surgery may be provided. In order to determine the desired size and shape of an improved titanium mesh implant, in 300, the size of a bony defect and/or void may be determined. This size may be determined prior to implant surgery taking place and may be accomplished in any of a variety of manners. For example, the size of the bony defect or void can be determined by a physical or visual inspection. Alternatively, a three-dimensional stereolithographic model of a patient's skull with an existing defect may optionally be obtained. If a three-dimensional model is made, the model may optionally include imaging and mapping of the area of the defect 302, imaging, mapping and/or modelling the surrounding bone 304, and mapping or modelling any pertinent nearby structures of critical importance like nerves, blood vessels, and/or sinus cavities.

Based on the size of the bony defect or void, an appropriately size implant may be chosen. As discussed below, implants may be generally sized so as to cover the most common sizes of bony defect of void in such a manner as they provide the desired neuroplastic effect along with minimal overlap on the bone surrounding the bony defect and/or void.

The selected implant may be a piece of “off-the-shelf” titanium mesh that may be treated, cut, or otherwise formed in any of a variety of manners. For example, the implant may be hand-bent and/or molded 312, cut 306, and/or otherwise formed pre-operatively such that it can fill and resolve the existing skull defect while also preserving any desired craniofacial symmetry with non-defect areas. It can be appreciated that, in general, human skulls have a substantially common radius of curvature. As such, when implants are formed, they can be bent or molded with such a radius of curvature. Further, it may be appreciated that when the implant, formed of titanium mesh, is cut in 306, it may be cut such that it has no square, squared, sharp or pointy edges. In other words, the implants may be formed such that they are rounded, with many being formed in a substantially circular or oval shape. Further, before or after cutting, the implants may be 308 or polished 310, as discussed in more detail below.

Once the appropriately sized implant is chosen, any further modifications may be performed 314 prior to the implant being surgically placed. It may be appreciated that the selection 305 of an appropriately sized implant may be such that no further modifications take place. This may be a result of the available implants being sized to provide desired coverage and protection of a bony void or defect while also having minimal overlap of the mesh on bone, so as to maximize the preoperative contour of the skull of a patient receiving an implant. However, depending on circumstances, further modifications, such as further bending or shaping, further cutting, or any other change desirable for the patient, may be performed 314 prior to or during implant surgery, as desired, appropriate, or necessary.

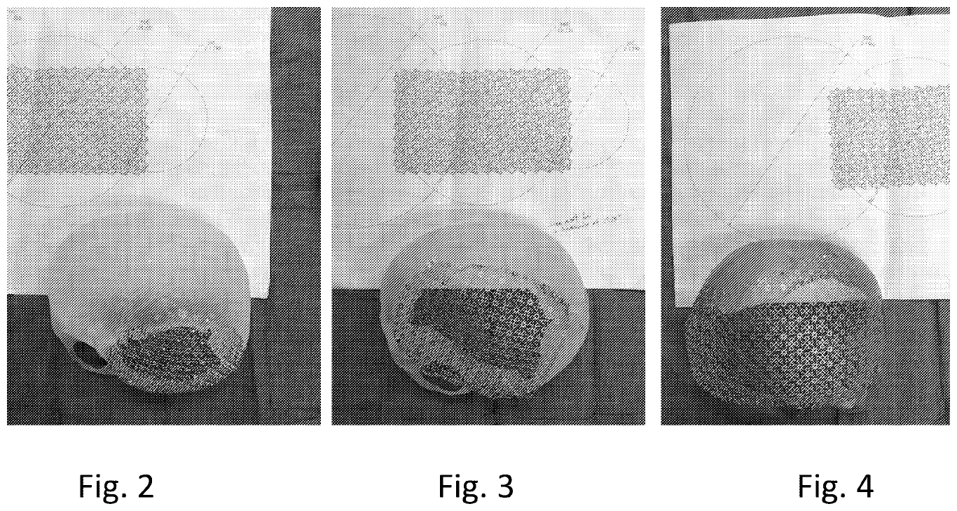

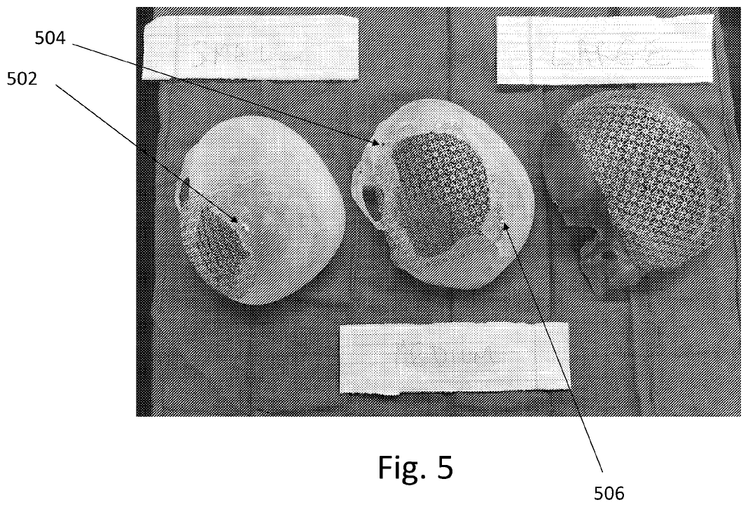

The titanium mesh implant may then be sterilized 316 for surgery and ultimately implanted during surgery 318 in rapid fashion, as opposed to the prolonged time periods necessary for alloplastic implants and thereby preventing the labor-intense and cost-intense aspects of 3D printed, custom titanium mesh. Exemplary views of craniofacial implants may be provided in FIGS. 2, 3, and 4, with a size comparison of the implants further shown in FIG. 5. These are discussed in more detail below.

The “off-the-shelf” or 3D printed titanium mesh craniofacial implant may have any of a variety of outcome-improving characteristics and complication-preventing qualities. For example, to improve its safety and quality, a frictionless, coating process may be applied 308 to make it more smooth and suitable for the constantly moving, mobile scalp and soft tissue above for long-term durability, and therefore improving on the current “sandpaper-like” titanium mesh products currently on the market. This coating, and/or manufacturing/finishing process which decreases the abrasiveness of the titanium mesh when coupled with the implant, allows for effective gliding to occur and decrease risk for eventual extrusion, infection, removal and failure—particularly in instances over thin scalps from repeated surgery or irradiation therapy for tumor disease. These unique qualities of a smoother, coated implant—avoiding repetitive injury from the sharp edges surrounding each little circle defect within—solve numerous problems associated with the prior art and provide enhanced durability and safety for the patient receiving the implant.

Further, the titanium mesh craniofacial implant may be polished 310 mesh such that it is smooth or not otherwise coarse. Thus, the titanium mesh craniofacial implant can perform in a desired and effective manner to protect the soft scalp and soft tissue (i.e. temporalis muscle and temporal fat pad) above the implant following implantation from long-term injury, eventual atrophy, and/or erosion leading to failure and removal. These qualities would represent significant improvements over the existing state of the art and enhancements to the treatment of patients.

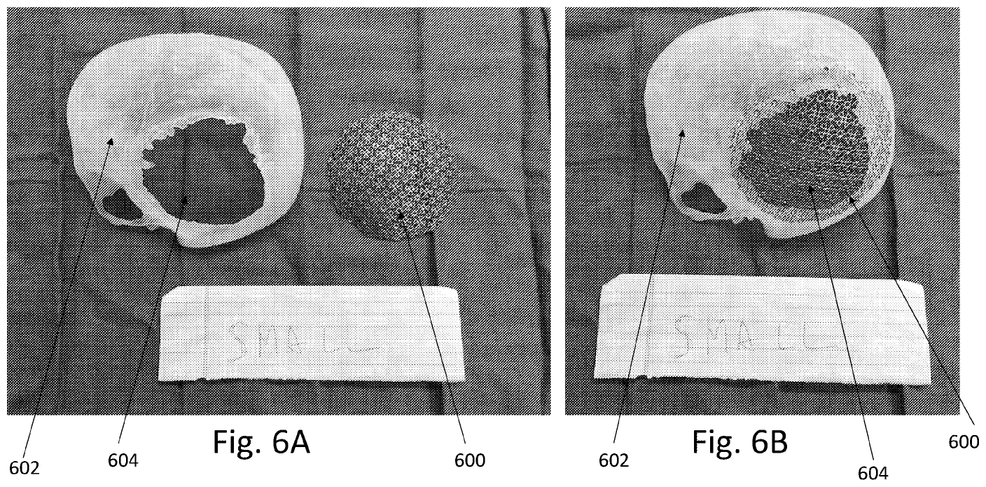

In a further embodiment, the titanium mesh craniofacial implant, whether 3-D printed or hand-bent to a stereolithographic model, can have mesh that is in a round, circular form instead of, for example, a square or rectangular form with unnecessary corners. As mentioned above, square or rectangular form mesh (or mesh otherwise having angles or sharp edges) of the prior art is known to always need cutting along its edges and corners during the implantation or otherwise during surgery because the defect or any other region being filled with the implant is never a square- or rectangular-shaped; the defects have any combination of rounded edges, sharp angles, and other anomalies that cannot be compensated for by the existing off-the-shelf implants. In FIGS. 6A and 6B, an exemplary implant 600 is shown with respect to a skull 602 having a void 604. While voids necessitating an implant are generally unique in appearance and in their edge qualities, void 604 provides an exemplary type of void to which an implant 600 formed of with the above method may be utilized and implanted. Further, it can be appreciated that the implant 600 has been molded or bent to substantially match the curvature of the skull 602, resulting in an implant and implant procedure that is safer for all parties, along with providing aesthetic benefits. Thus, unlike the prior art where the further forming and shaping of an implant during surgery adds unjustifiable risk of an iatrogenic glove or skin injury to the medical personnel during the surgery due to the sharp edges of the square or rectangular mesh, adds prolonged anesthesia time and morbidity to the surgery (i.e. extra time asleep), and results in wasted material (such as the material that is cut away during implantation). Further, the overwhelming majority of skull defects are circular or oval (as seen in the example of FIG. 6), not square- or rectangular-shaped, and thus the titanium mesh craniofacial implant formed in a rounded, oval-like fashion provides for an improvement over the existing implants and related uses and implementations. In a further exemplary embodiment, the titanium mesh craniofacial implant can be pre-cut so as to remove corners which adds a variety of cost-saving benefits and safety features over the current state of the art, including reduced surgical time and improved safety.

Additionally, the titanium mesh craniofacial implant can be pre-formed in a variety of sizes most-applicable to the common skull defect sizes, as opposed to the prior art method where randomly selected dimensions are used, for example “small”, “medium”, and “large”, as shown in FIGS. 2-5, would refer to known, average skull defect sizes found within skull implant databases and determined through empirical evidence. In practice, it has been found that most bony defects or voids fall into a range of about 5-10 cm (small), about 10-15 cm (medium) and about 15-20 cm (large). A large size implant, as shown in exemplary FIG. 5, may be formed so as to substantially cover half of a skull. Further noted above, it may be desirable to have a minimal amount of overlap of the titanium mesh of the implant on the bone of the patient, thus an implant can be selected accordingly based on the typical size and dimensions of small, medium or large. In some circumstances, for example where a bony void or defect is smaller than the “small” size, a surgeon or other appropriate party can resize, cut, or trim the implant based on the needs of the patient either before or during surgery. Alternatively, in situations where there is a bony defect that is larger than the “large” size, two or more implants may be utilized together, for example two large implants, a large and a small implant, etc. It may further be appreciated in situations where two (or more) implants may be desired or necessary, the two implants may be secured to the bone and each other such that there is no mobility between the implants, as mobility between or among the implants can have adverse effects on a patient.

Thus, following the three-dimensional modelling of the skull and skull defect, an appropriately sized titanium mesh craniofacial implant can be chosen (for example small, medium, or large) and modified for the patient as appropriate for that particular patient. Having varied, pre-formed titanium mesh implants with both accurate pre-cut surface areas, an absence of unnecessary corners, and pre-bent curvatures for optimal temporal/soft tissue contouring drastically reduces the time needed to make a titanium mesh craniofacial implant, reduce waste, improves the safety and durability of the implant, and reduces personal injury during the hand-cutting process of the implant itself. In some examples, 20-30 minutes of time may be saved from the prior art methods of manipulating, cutting, and shaping the implant to fit a patient and remove sharp edges, with some of the time savings taking place during surgery, representing significant advances in both safety and cost, as preventing injury is one of the highest priorities in the operating room. Further, given the rise of the coronavirus-based pandemic, the improved safety of the cranial implant and its associated surgery will help control virus transmission during such procedures. As a result, removing corners from a square or rectangular patient specific mesh implant, as a simple but powerful method to prevent iatrogenic injury, would be a major advance in this field.

Further, the titanium mesh craniofacial implant can be pre-bent prior to implantation. This can be desirable because there are typically no flat-shaped skull defects in a human given the cranium's radius of curvature. Thus, having titanium mesh implants supplied with a flat shape only mandates additional effort and time in the operating room, which are undesirable from safety, time, and cost perspectives, as described above. The pre-bending of the titanium mesh craniofacial implant during the fabrication process can thereby reduce surgeon effort before and during surgery, reduce the amount of associated artistry involved in properly forming and modelling an craniofacial implant so as to achieve the desired remediation of the defect and preserve craniofacial symmetry using surgical know-how related to addressing contour irregularities associated with cranioplasty reconstruction, and, ultimately, result in an improved implant that has improved functionality in exposure-prone areas and also improved post-operation appearance on the recipients of the implant.

In another exemplary embodiment, circular holes inside the polished, pre-bent, pre-cut titanium mesh craniofacial implant can have countersinking as to allow a much lesser or shallower screw profile during inset. As shown in exemplary FIG. 5, the countersinking of screw holes 502, 504, and 506, for example, may be utilized in any desired area of an implant to secure the implant to bone. For example, with a typical screw for neuroplastic implants having a length of 4 mm, where 2 mm is inserted into bone and 2 mm is in the mesh or implant, countersinking can provide for a decrease of 1 mm or more. In other words, with countersinking, 3 mm of a 4 mm screw can be inserted into bone with only 1 mm in the mesh or implant. The result can provide a flat implant profile. Thus, the countersinking along the mesh implant can improve outcomes with a smooth or smoother contour of the craniofacial implant since having flat-head screws inside countersunk areas provides the ultimate low-profile construct. Small screw edges underneath a thin or atrophied scalp, particularly with smokers or radiated patients, can be quite problematic and include symptoms such as chronic pain, visible deformity, and may even lead to extrusion/infection necessitating the removal of mesh implant, all of which is decreased with the countersinking.

In a further embodiment, the mesh of the titanium mesh craniofacial implant can be asymmetrical. The asymmetrical mesh may provide for additional angles for bending and shaping the implant, leading to easier, less time consuming, and more accurate implant structures. Thus, there are associated benefits related to enhanced appearance and craniofacial symmetry for the recipient of the implant, decreased preparation and surgery times, and lower costs when compared with traditional symmetrical mesh patterns. Also, by modifying the standard circular hole design of titanium mesh, one can strategically minimize the number of sharp edges associated with each little circle, and thereby improve its safety and long-term durability.

In another exemplary embodiment during the implantation or surgery, the titanium mesh craniofacial implant can be placed on top of the temporalis muscle in an effort to prevent disruption of neo-angiogenesis and unnecessary bleeding. Such placement of the titanium mesh craniofacial implant may provide for desired reconstruction of the temporal area as well as the missing bone in the skull defect, which is especially relevant in instances of secondary cranioplasty following head trauma or tumor requiring craniectomy and staged surgery to follow. During this time interval between the two surgeries, the temporalis muscle scars down to the brain and suffers atrophy thereby contributing to temporal hollowing deformity. In addition, the titanium mesh craniofacial implant can be provided with a polished platinum-like appearance or other shimmering, aesthetically pleasing appearance. This polished appearance will not only be favorable in the operating room, but will also provide a much needed safety feature as a unique method to prevent or decreased overlying soft tissue abrasion or injury.

The foregoing description and accompanying figures illustrate the principles, preferred embodiments and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

1. A method of forming a craniofacial implant for neuroplastic surgery, comprising:
determining a size of a bony defect and/or void in a craniofacial region;
selecting an implant formed of a piece of pre-manufactured titanium mesh of a size that covers the bony defect and/or void and at least a portion of the bone surrounding the bony defect and/or void, wherein the implant is pre-sized to common craniectomy dimensions and pre-contoured using a normal radius of curvature for a human skull; and
sterilizing the implant.

2. The method of forming a craniofacial implant for neuroplastic surgery of claim 1, further comprising polishing the titanium mesh after it is cut.

3. The method of forming a craniofacial implant for neuroplastic surgery of claim 1, further comprising coating the titanium mesh after it is cut, wherein the coating comprises a low friction coating that adheres to the titanium mesh.

4. The method of forming a craniofacial implant for neuroplastic surgery of claim 3, wherein the coating provides the titanium mesh with a smooth exterior and substantially prevents micro-injury in an implant area.

5. The method of forming a craniofacial implant for neuroplastic surgery of claim 1, further comprising molding the implant by hand using a stereolithographic model.

6. The method of forming a craniofacial implant for neuroplastic surgery of claim 1, further comprising cutting and molding the titanium mesh by a three-dimensional printer.

7. The method of forming a craniofacial implant for neuroplastic surgery claim 1, further comprising forming one or more circular holes with countersinking in the implant.

8. The method of forming a craniofacial implant for neuroplastic surgery of claim 1, further comprising generating a three-dimensional model of the craniofacial region having the void and/or bony defect.

9. The method of forming a craniofacial implant for neuroplastic surgery of claim 8, further comprising mapping the bony defect and/or void, as well as the bone surrounding the bony defect and/or void in the craniofacial region.

10. The method of forming a craniofacial implant for neuroplastic surgery of claim 1, wherein the implant is round with no edges or corners.

11. The method of forming a craniofacial implant for neuroplastic surgery of claim 1, wherein the titanium mesh implant is one of 5-10 cm, 10-15 cm, and 15-20 cm in length.

12. The method of forming a craniofacial implant for neuroplastic surgery of claim 1, further comprising coupling a second titanium mesh implant to the implant to cover a bony void and/or defect greater than 20 cm in length.

13. A craniofacial implant for neuroplastic surgery, comprising:
a piece of titanium mesh, the titanium mesh formed so as to be substantially round or otherwise without corners, the titanium mesh molded and shaped to substantially match the radius of curvature of a human skull, the titanium mesh further comprising a smooth surface and a plurality of screw holes formed with countersinking.

14. The craniofacial implant for neuroplastic surgery of claim 13, wherein the piece of titanium mesh has a length in a range of 5 cm-10 cm.

15. The craniofacial implant for neuroplastic surgery of claim 13, wherein the piece of titanium mesh has a length in a range of 10 cm-15 cm.

16. The craniofacial implant for neuroplastic surgery of claim 13, wherein the piece of titanium mesh has a length in a range of 15 cm-20 cm.

17. The craniofacial implant for neuroplastic surgery of claim 13, wherein the piece of titanium mesh is coated with a low friction coating.
